(19) United States
(12) Patent Application Publication
(10) Pub. No.: US 2011/0040357 A1
(43) Pub. Date: Feb. 17, 2011

(54) COILED LIGHT DIFFUSER FOR IRRADIATION OF BIOLOGICAL TISSUE WITH LIGHT BEAM AND LIGHT-DIFFUSING DEVICE COMPRISING THE SAME

(75) Inventors: Tsunenori Arai, Kanagawa (JP); Nozomi Morita, Kanagawa (JP); Koichi Sakai, Tokyo (JP)

Correspondence Address: FOLEY AND LARDNER LLP;SUITE 500, 3000 K STREET NW, WASHINGTON, DC, 20007, US

(73) Assignees: Keio University; Yokowo Co., Ltd.

(21) Appl. No.: 12/936,606

(22) PCT Filed: Apr. 7, 2009
(86) PCT No.: PCT/JP2009/057130

§ 371 Date: Oct. 6, 2010

(30) Foreign Application Priority Data
Apr. 7, 2008 (JP) 2008-099702

(51) Int. Cl. A61N 5/06; G02B 5/02

(52) U.S. Cl. 607/88; 359/599

(57) ABSTRACT

According to the present invention, a light diffuser capable of efficiently and uniformly irradiating a wide area of a biological tissue with a light beam such as a laser light beam, which can be readily produced at low cost, and a light-diffusing device comprising the same are provided.

Specifically, a coiled light diffuser consisting of a portion for attachment to an optical fiber and a portion for diffusing light transmitted from an optical fiber, which is attached to a light transmission end of an optical fiber for diffusing light transmitted from the optical fiber in a direction differing from the light transmission direction, and which is formed by coiling a wire rod at least the surface of which comprises a light-reflective metal into a cylindrical shape for the irradiation of a biological tissue with a light beam, and a light-diffusing device to which the coiled light diffuser is attached are provided.

### TECHNICAL FIELD

The present invention relates to a technique for treating a biological tissue lesion by irradiating a living body with a light beam such as a laser light beam: Specifically, the present invention relates to a light diffuser for widely irradiating a biological tissue by diffusing irradiated light and also relates to a light-diffusing device.

### BACKGROUND ART

A light beam such as a laser light beam is used for treatment, including photochemical treatment of biological tissue, biological tissue adhesion, prevention of post-percutaneous transluminal coronary angioplasty restenosis in the cardiovascular system, and myocardial tissue ablation for treatment of arrhythmia and other diseases (see Patent Documents 1 to 4). For instance, photochemical treatment comprises administering a photosensitizer into a lesion such as a cancer tissue lesion and irradiating the tissue with laser light to destroy the lesion. In addition, in the case of aortic dissection, dissected layers can adhere to each other when the dissected lesion is irradiated with laser light.

Upon irradiation of a biological tissue with laser light, if a biological tissue to he treated is located inside a living body, a catheter comprising an optical fiber capable of transmitting a light beam is used in such a manner that the optical fiber is inserted into a narrow hollow organ such as a digestive organ or a blood vessel and a portion for irradiating a light beam of the optical fiber is placed in the vicinity of the biological tissue, followed by light beam irradiation. When an optical fiber is used for light beam irradiation, a light beam is irradiated from a thin optical fiber tip so as to make it possible to irradiate a narrow area alone with light. In addition, upon laser treatment for a narrow hollow organ, a laser light is transmitted in a diagonal direction (tangential direction) with respect to a lesion. This causes problems in terms of the absolute value of irradiation dose and uniformity of irradiation.

In order to solve the above problems regarding in vivo treatment involving light beam irradiation, techniques for diffusing light transmitted from an optical fiber tip so as to irradiate a wide area have been developed (see Non-Patent Document 1 and Patent Document 5). For example, there is a technique for processing a light transmission portion of an optical fiber so as to diffuse a light beam (see Patent Document 5).
Patent Document 1: WO2004/112902
Patent Document 2: WO2005/079690
Patent Document 3: JP Patent Publication (Kokai) No. 2006-149974 A
Patent Document 4: JP Patent No. 3739038
Patent Document 5: JP Patent Publication (Kokai) No. 2001-204831 A
Non-Patent Document 1: Leonid Vellclov et al., APPLIED OPTICS, Vol. 44, No. 14, 10 May 2005, pp. 2754-2758

### DISCLOSURE OF THE INVENTION

### Problem to be Solved by the Invention

It is an object of the present invention to provide a light diffuser capable of efficiently and uniformly irradiating a wide area of a biological tissue with a light beam such as a laser light beam, which can be readily produced at low cost, and a light-diffusing device comprising the same.

### Means for Solving Problem

As described above, there are conventional techniques for in vivo treatment involving light beam irradiation, which comprise diffusing a light beam transmitted from an optical fiber tip so as to irradiate a wide area with the light beam. For example, there is a light diffusion and irradiation device obtained by processing an optical fiber tip so as to form many grooves on the side face thereof However, conventional techniques are problematic in terms of processing difficulties and high production costs. Due to high production costs, such devices cannot be used in a disposable form. Therefore, new problems regarding the application of appropriate sterilization processes, the securement of durability, and the like have arisen, causing safety-related problems in many cases. In addition, it has been difficult to irradiate an arbitrary in vivo tissue such as a vascular tissue having a complex structure with a light beam due to lack of device flexibility.

The present inventors conducted intensive studies on the development of a light diffuser with high light diffusion efficiency that can be readily produced at low cost. As a result, they conceived of processing a wire rod capable of reflecting light on the surface thereof by coiling it into a coiled shape and attaching the thus obtained coiled light diffuser made up of the wire rod to an optical fiber tip. The present inventors found that a light beam transmitted into the light diffuser that has been attached to an optical fiber tip illuminates the surface of the wire rod processed into a coiled shape and is reflected therefrom such that light is diffused from spaces between neighboring coils of the wire rod, resulting in uniform irradiation of a wide area of a biological tissue in the vicinity of the light diffuser. Further, the present inventors found that such light diffuser made up of an elastic wire rod can be safely used because it can bend to fit into a portion with a complex structure in a living body.

The present inventors further found that the area irradiated with diffused light can be controlled by changing the pitch of a coiled wire rod or the numerical aperture (NA) of an optical fiber. Accordingly, the present inventors produced a coiled light diffuser capable of diffusing light transmitted from an optical fiber so as to widely and uniformly irradiate a biological tissue with light circumferentially in the light irradiation direction for in vivo treatment using a light beam. This has led to the completion of the present invention.

Specifically, the present invention is described below.
[1] A coiled light diffuser consisting of a portion for attachment to an optical fiber and a portion for diffusing light transmitted from an optical fiber, which is joined or detachably attached to a light transmission end of an optical fiber for diffusing light transmitted from the optical fiber in a direction differing from the light transmission direction, and which is formed by coiling a wire rod at least the surface of which comprises a light-reflective metal into a cylindrical shape for the irradiation of a biological tissue with a light beam.
[2] The coiled light diffuser according to [1], wherein coils of the wire rod that constitute the portion for diffusing light transmitted from an optical fiber are spaced at constant pitches.
[3] The coiled light diffuser according to [1], which is designed such that coils of the wire rod that constitute the portion for diffusing light transmitted from an optical fiber are spaced at variable pitches, provided that coils are narrowly spaced on the butt side and widely spaced on the tip side.
[4] The coiled light diffuser according to any one of [1] to [3], which comprises a metallic wire rod or a wire rod the surface of which is coated with a light reflective metal.
[5] The coiled light diffuser according to any one of [1] to [4], which is covered with a light-permeable resin film for the prevention of body fluid infiltration.
[6] The coiled light diffuser according to any one of [1] to [5], wherein the tip of the coiled light diffuser accommodates a metal marker used for X-ray radioscopy and/or a biopotential measurement electrode.
[7] A light-diffusing device, which comprises the coiled light diffuser according to any one of [1] to [6].
[8] An optical fiber for in vivo treatment, to the tip of which the coiled light diffuser according to any one of [1] to [6] is attached.

### Effects of the Invention

The coiled light diffuser and the light-diffusing device comprising the same of the present invention can be used for treatment, including photochemical treatment of biological tissue, adhesion of biological tissue, prevention of post-percutaneous transluminal coronary angioplasty restenosis in the cardiovascular system, and myocardial tissue ablation for treatment of arrhythmia and other diseases. The diffuser and the device can efficiently and uniformly irradiate a wide area of a lesion to be treated with a light beam such as a laser light beam. In addition, the coiled light diffuser of the present invention can be readily produced at low cost because it can be produced by processing (coiling) a metal wire rod or the like into a coiled shape. It can be safely used in vivo as a disposable element. Further, since the technology for processing a wire rod into a coiled shape has been developed, such coiled light diffuser can he produced by freely determining the size of a light diffuser in terms of, for example, the pitch of neighboring coils of a wire rod. Accordingly, the area subjected to irradiation with diffused light can be freely controlled depending on purpose.

This description includes part or all of the contents as disclosed in the description and/or drawings of Japanese Patent Application No. 2008-099702, which is a priority document of the present application.

### BRIEF DESCRIPTION OF THE DRAWINGS

FIGS. 1A and 1B show the definitions of the sizes of various portions of the coiled light diffuser of the present invention. FIG. 1A shows a dextrorse coiled light diffuser and FIG. 1B shows a sinistrorse coiled light diffuser.

FIG. 2A shows an example of the coiled light diffuser of the present invention. The coiled light diffuser in the figure has a portion for attachment to an optical fiber capable of transmitting light and a portion for diffusing transmitted light. The portion for diffusing transmitted light is a variable-pitch portion. FIG. 2B shows an optical fiber with the coiled light diffuser of the present invention attached thereto.

FIG. 3 illustrates the numerical aperture (NA) of an optical fiber.

FIG. 4 shows the direction of irradiation of light diffused by the coiled light diffuser of the present invention.

FIG. 5 shows an experimental system used for diffused light measurement in the Examples.

FIG. 6 shows a distribution of intensities of light diffused in the incident direction of a laser beam at different pitches for constant-pitch coiled light diffusers.

FIG. 7 shows a distribution of intensities of light diffused in the incident direction of a laser beam at different pitches for variable-pitch coiled light diffusers.

FIG. 8 shows a distribution of intensities of light diffused in the incident direction of a laser beam at different NA values for the laser beam

### BEST MODE FOR CARRYING OUT THE INVENTION

Hereinafter, the present invention is described in detail. The present invention relates to a light diffuser for diffusing a light beam transmitted from an optical fiber such that a wide area of a biological tissue to be treated is uniformly irradiated with the light beam for treatment of a biological tissue with a light beam. When light is simply transmitted from an optical fiber, light irradiation takes place with light spreading to a certain extent only in the transmission direction. In this case, a narrow area of a biological tissue can be irradiated. In addition, irradiation with a light beam within a narrow hollow organ results in tangential irradiation, causing problems regarding the light irradiation dose and the light irradiation uniformity. In such case, high treatment effects cannot always he obtained. The use of the coiled light diffuser of the present invention allows a light beam transmitted from an optical fiber to be circumferentially diffused by the light diffuser not only in the transmission direction but also in a direction orthogonal to the transmission direction (i.e., in the circumferential direction), resulting in irradiation of a surrounding biological tissue. According to the present invention, regarding the direction of irradiation with diffused light, the direction of light transmitted from an optical fiber refers to the z axis direction and the circumferential direction with respect to the transmission direction refers to the a direction. Specifically, the use of the coiled light diffuser of the present invention allows a light beam transmitted from an optical fiber to be diffused not only in the z axis direction but also in the e direction, resulting in wide and uniform irradiation of a biological tissue. It is possible to learn whether or not a biological tissue can be widely and uniformly irradiated with a light beam transmitted from an optical fiber by, for example, examining the distribution of intensities of light diffused in the z axis direction. Regarding the distribution, the horizontal axis is designated as representing the distance from the fiber end surface (from which a light beam is transmitted) to a given point located along the z axis direction and the vertical axis is designated as representing the diffused light intensity at a given point located a fixed distance away from the fiber centerline.

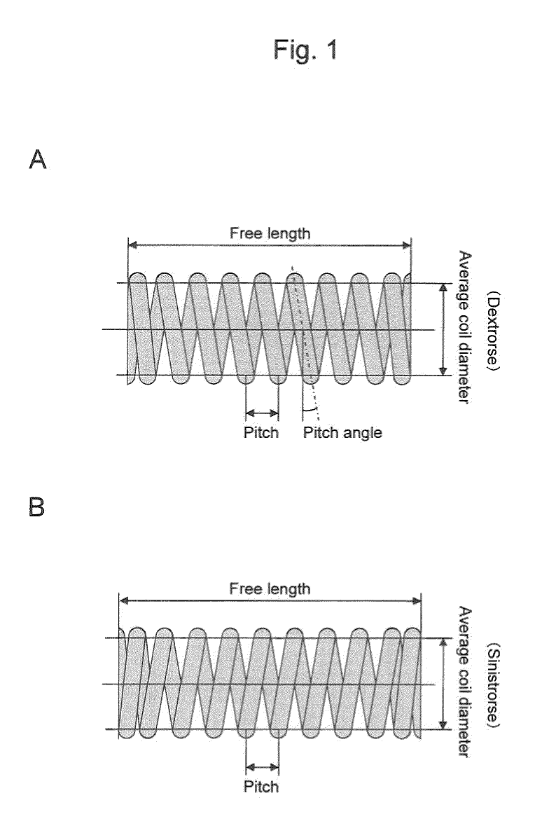

The light diffuser of the present invention has a coiled shape; that is to say, a spiral shape. It has a shape similar to a coiled spring shape and preferably a cylindrical coiled shape. Specifically, it has a shape similar to the shape of a cylindrical coiled spring formed by coiling a wire rod into a cylindrical shape; that is to say, a spiral shape. In the descriptions below of the individual portions or the sizes thereof of the light diffuser of the present invention, since the shape of the light diffuser is similar to a coiled spring shape, the terms generally used for coiled springs are used as terms specifying individual portions and sizes of the light diffuser (FIGS. 1A and 1B). Note that even if the terms used for coiled springs are used herein or even if the coiled light diffuser of the present invention is used while in a bent state because of its elasticity, the coiled light diffuser of the present invention is not intended to be used as a spring. In this regard, it is an element that should be distinguished from a spring.

The coiled light diffuser of the present invention may be formed by dextrorsely (FIG. 1A) or sinistrorsely (FIG. 1B) coiling a wire rod.

The free length (free height) of the coiled light diffuser of the present invention can be adequately determined depending on the type of biological tissue to be irradiated with a light beam. However, it is preferably 10 to 200 mm and more preferably 20 to 100 mm. For instance, when the inside of a blood vessel is irradiated with a light beam, it is 20 to 100 mm. The coil outer diameter of the coiled light diffuser can be adequately determined depending on the type of biological tissue to be irradiated with a light beam. However, it is preferably 0.2 to 5 mm and more preferably 0.5 to 3 mm. For instance, when the inside of a blood vessel is irradiated with a light beam, it is 0.5 to 3 mm. In addition, desirably, a constant coil outer diameter is maintained; however, it may vary in the length direction. For instance, the coiled light diffuser may have a tapered structure in which the outer diameter gradually decreases or increases from the portion for attachment to an optical fiber to the tip of the diffuser.

The cross-sectional shape of a wire rod constituting the coiled light diffuser of the present invention is not limited. It may have a different shape, such as a round shape (e.g., true circle or ellipse), a semicircular shape, or a polygonal shape (e.g., triangle, rectangle, pentagon, or hexagon). The cross-sectional shape of a wire rod can be selected in accordance with the diffusion direction or the reflection efficiency when light is transmitted from an optical fiber and illuminates the surface of the coiled light diffuser of the present invention so as to be reflected or to be diffused. At such time, the cross-sectional shape of a wire rod can be designed based on the fact that the incidence angle is equivalent to the reflection angle upon reflection of a light beam transmitted from an optical fiber from a coiled light diffuser. The wire rod size in terms of the diameter (of a linear wire rod) is 0.02 to 0.5 mm and preferably 0.05 to 0.3 mm when the section shape is a round shape, for example. In addition, the total number of coils of a wire rod is 4 to 10000 and preferably 8 to 7000.

When the cross-sectional shape is a rectangle, the wire rod width is 0.02 to 3 mm while the wire rod thickness is 0.02 to 0.5 mm. In this case, a wire rod is coiled such that wide faces thereof serve as the inner and outer surfaces of coils. More preferably, the wire rod width is 0.05 to 2 mm while the wire rod thickness is 0.05 to 0.3 mm. In addition, the total number of coils made of a wire rod is 4 to 10000 and preferably 4 to 6000.

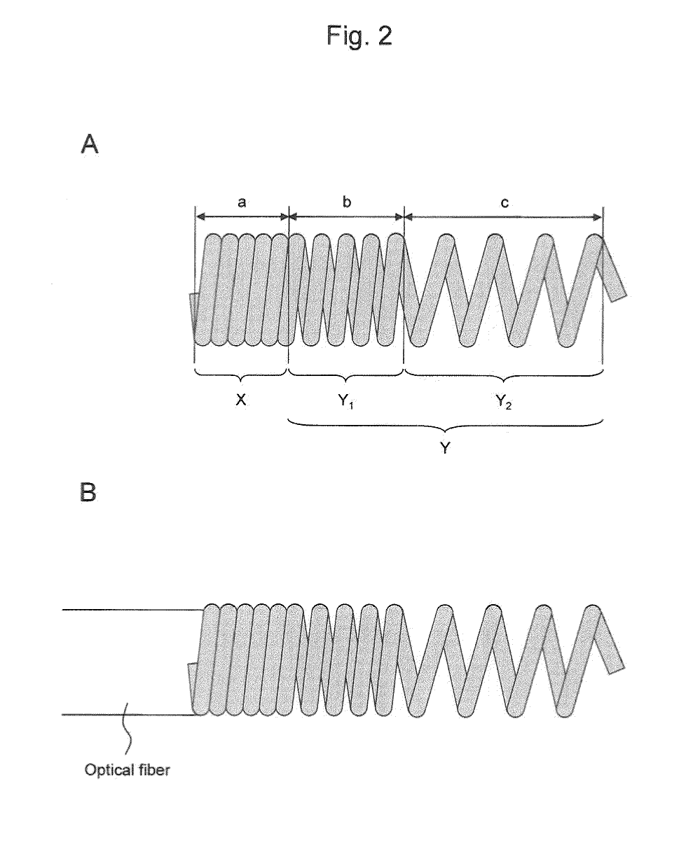

The coiled light diffuser of the present invention continuously has a portion for attachment to an optical fiber capable of transmitting light and a portion for diffusing transmitted light. FIG. 2A shows the coiled light diffuser of the present invention. In FIG. 2A, the portion denoted by X corresponds to a portion for attachment to an optical fiber capable of transmitting light and the portion denoted by Y corresponds to a portion for diffusing transmitted light. The portion for attachment to an optical fiber capable of transmitting light functions to fix the coiled light diffuser of the present invention to an optical fiber. The portion for diffusing transmitted light comprises a material that can reflect a light beam and has spaces through which a diffused light beam passes. Therefore, it functions to diffuse a light beam transmitted from an optical fiber into the light diffuser for light emission to the outside. In FIG. 2A, the letters a, b, and c each denote the length of a relevant portion. FIG. 2B shows the coiled light diffuser of the present invention attached to an optical fiber.

The portion for attachment to an optical fiber is formed on one end of a coiled light diffuser. For instance, this portion is formed as a tightly coiled portion on one end of the coiled light diffuser. Such tightly coiled portion is a coil portion in which each interval (pitch) between neighboring coils formed on one end of the coiled light diffuser is adjusted to substantially zero. The portion for attachment to an optical fiber capable of transmitting light is attached (e.g., fitted) onto the tip of an optical fiber. The coiled light diffuser may be fixedly joined in a non-detachable manner or attached in a detachable manner to the tip of an optical fiber. The length in the free length direction (denoted by “a” in FIG. 2A) of the portion for attachment to an optical fiber capable of transmitting light is 1% to 50% (the percentage of the total length “a+b+c” represented by the length “a” in FIG. 2A) and preferably 1.5% to 30% of the free length. The actual length thereof would vary depending on the type of biological tissue to be irradiated with a light beam. It is 0.1 to 100 mm and preferably 0.15 to 30 mm. FIG. 1B shows an optical fiber to which the coiled light diffuser of the present invention is attached. In addition, the portion for attachment to an optical fiber capable of transmitting light may be a cylindrical portion made of metal, or the like. In such case, the cylindrical portion can be attached to the tip of an optical fiber. The cylindrical portion and the coiled portion formed by coiling a wire rod can he joined by welding or the like so as to he integrated.

The coiled light diffuser of the present invention can be attached to the tip of an optical fiber with the use of an adhesive or via welding.

The portion for diffusing transmitted light of the coiled light diffuser of the present invention (portion Y in FIG. 2A) circumferentially diffuses light from between neighboring coils such that a surrounding biological tissue is irradiated with light. If the pitch of the portion is narrow, a light beam transmitted from an optical fiber becomes less likely to be diffused from between neighboring coils. In this case, the light beam can illuminate the tip of the light diffuser so as be diffused, allowing irradiation of a wider area of biological tissue. The term “pitch” used herein refers to the distance between the centers (located on a line parallel to the centerline of the coiled light diffuser) of material sections of neighboring coils on a cross section along the centerline of the coiled light diffuser (FIG. 1A). For example, the pitch for the portion for diffusing transmitted light is 0.005 to 1.5 mm and preferably 0.01 to 0.75 mm. When a round wire with a wire diameter of 0.02 to 0.5 mm is used, the pitch is 110% to 500% and preferably 130% to 300% of the wire diameter of the wire rod. In addition, when a wire rod having a rectangular cross-sectional shape is used, the pitch is 110% to 500% and preferably 130% to 300% of the width of the wire rod.

The portion for diffusing light may be a constant pitch (uniform pitch) portion. In other words, neighboring coils of a wire rod may be regularly spaced within the portion for diffusing light. In view of uniform irradiation of a wide area of biological tissue with diffused light, it is desired that the portion be a variable pitch (non-uniform pitch) portion. In the portion for diffusing light of the coiled light diffuser of the present invention, coils are tightly formed and spaced at narrow pitches on the side for attachment to an optical fiber (i.e., the side adjacent to the portion for attachment to the optical fiber) while coils are loosely formed and spaced at wide pitches in a portion positioned away from the portion for attachment to an optical fiber. Specifically, the portion for diffusing light can be designed such that coils are narrowly spaced on the butt side and widely spaced on the tip side. The term “the butt side” used herein refers to the side for attachment to an optical fiber. Here, it is also possible to gradually or continuously extend narrow pitches to result in wide pitches. In this case, the pitch is gradually increased from the portion attached to an optical fiber to the tip portion. In addition, the above portion may consist of a constant-narrow pitch portion and a constant-wide pitch portion. In the case of any variable-pitch coiled light diffuser, neighboring coils located nearest to an optical fiber are spaced at narrower pitches than neighboring coils located farthest from the optical fiber. For example, FIG. 2A shows an example of the coiled light diffuser of the present invention which consists of a constant-narrow pitch portion and a constant-wide pitch portion. In FIG. 2A, the portion denoted by Yl corresponds to a portion comprising coils tightly formed at narrow pitches and the portion denoted by Y2 corresponds to a portion comprising coils loosely formed at wide pitches. The length b of Y1 is, for example, 5% to 50% and preferably 10% to 35% of the length b+c of Y. In addition, the pitch in portion Y1 is 10% to 50% and preferably 20% to 45% of the pitch in portion Y2.

When the portion for diffusing transmitted light of a coiled light diffuser is a variable-pitch portion, a light beam transmitted into a coiled light diffuser from an optical fiber is not excessively diffused outside the light diffuser from a narrow-pitch portion. Accordingly, the light beam can reach the tip of the light diffuser so as to be diffused. Therefore, a wider area of biological tissue can be irradiated.

The pitch angle of the coiled light diffuser of the present invention is 5 to 70 degrees and preferably 14 to 60 degrees.

A wire rod constituting the coiled light diffuser of the present invention reflects light on the surface thereof and comprises a light reflective substance (capable of reflecting light) at least on the surface thereof. Examples of a substance capable of reflecting light include inorganic substances such as metals, glass, mica, and silica. A wire rod in its entirety may be formed from a single substance. The coiled light diffuser may he obtained by forming a linear material that can serve as a matrix into a coiled shape and coating the surface of the coiled material with a substance capable of reflecting light. In some cases, the coiled light diffuser of the present invention is used in a bent state while being in contact with tissue in a living body. Therefore, it is desirable for a substance serving as a matrix to have elasticity such that the tip of the obtained product can be bent when inserted into a complex lumen tissue.

Specifically, the coiled light diffuser in its entirety may he formed from a metal. It may be formed from a substance such as a resin and the surface thereof may he coated with a metal. Further, the surface may be coated with a metal powder so as to form a metal film thereon. For formation of a metal thin film on such surface, a method involving plating, sputtering, evaporation, or the like can be carried out. In addition, a thick film can be formed by electroless nickel-phosphate plating (film thickness: 5 to 20 μm, for example). If a wire rod serving as a matrix has a concave-convex surface, it is preferable to form a metal thick film by the above method. As a metal to be used, a highly light reflective metal is preferable. Examples thereof include gold, platinum, stainless steel (e.g., SUS 304 or SUS 316), aluminum, tantalum, nickel, tungsten, copper, brass, piano wire, nickel titanium, and alloys thereof. Of these, for example, stainless steel and a platinum alloy are preferable in view of light reflectance, moldability, shape retainability, in vivo affinity, cost, and the like. In addition, the wire rod surface may be treated so that it becomes shiny such that specular reflection of light takes place thereon or it may he treated so that it becomes partially rough such that diffuse reflection of light takes place thereon. However, in order to securely form a practically useful light diffusion area in the z axis direction, it is preferable to treat the surface such that specular reflection takes place. According to the invention of the present application, a phenomenon in which light transmitted from an optical fiber illuminates a coiled light diffuser and reflects in a direction other than the transmission direction is explained by noting that light is diffused. Such diffused light includes light derived from specular reflection and light derived from diffuse reflection.

The coiled light diffuser of the present invention can be produced using a coiling machine, for example. In addition, it can be formed by MIM (metal injection molding). MIM is a method for obtaining a single metal molded product wherein a resin mixed with a metal powder is subjected to injection molding and the resin is completely removed via sintering. The method is appropriate for the molding of a light diffuser having a complex shape. If a wire rod comprises a resin but not a metal, the coiled light diffuser is preferably produced by injection molding.

The coiled light diffuser of the present invention may comprise a marker and/or a biopotential measurement electrode for monitoring the position of a light beam transmission portion. As such marker, a maker used for X-ray radioscopy can be used. Under observation by X-ray radioscopy from the external side, the position where the tip portion of the coiled light diffuser is located determined, allowing the placement of the coiled light diffuser at an appropriate treatment portion. As an X-ray impermeable marker, an X-ray impermeable metal can be used. In terms of in vivo affinity, platinum, gold, iridium, tungsten, tantalum, and alloys thereof can be preferably used. Alternatively, a resin mixed with a powder of such a metal can be used. For instance, the above marker can be attached to a tightly coiled portion formed on the end opposite to the end that is attached to an optical fiber of the coiled light diffuser.

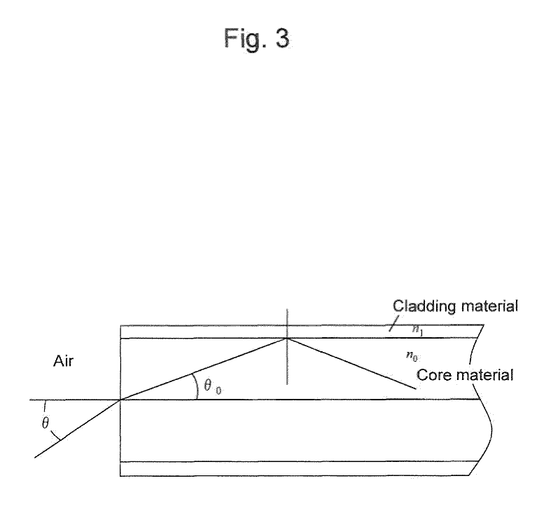

The light irradiation area and the light irradiation uniformity for light diffused from the light diffuser can be controlled based on the numerical aperture (NA) of a transmitted beam from the optical fiber. The numerical aperture of a transmitted beam can be changed based on the light receiving angle (θ) at the inlet of an optical fiber. The upper limit of the NA can be determined based on a numerical aperture that allows light transmission by an optical fiber. As shown in FIG. 3, a light beam is incident upon an optical fiber at the angle θ (light receiving angle) and propagated in the fiber. In such case, nsin θ is referred to as the “fiber NA.” Herein, the letter “n” refers to the refractive index in an external environment in which a fiber is placed. Herein, “n” is 1 when the fiber is used in the air. When the fiber is used in liquid such as blood, “n” refers to the liquid refractive index. The NA can be controlled by changing the angle at which light is received from a fiber. When the NA increases, the area irradiated with diffused light decreases inversely. When the NA decreases, the area irradiated with diffused light increases inversely. This is because the smaller the NA, the shallower the angle of a light beam transmitted into a coiled light diffuser (in the tangential direction), allowing the transmitted light beam to travel inside the light diffuser so as to reach the tip of the light diffuser without being leaked outside the light diffuser. The NA is 0.01 to 1.0 and preferably 0.02 to 0.4. The NA is determined depending on the refractive index of a core material constituting an optical fiber and that of a cladding material that covers the core. Therefore, an optical fiber with a specific NA can be selected. Alternatively, an arbitrary NA value can be determined by, for example, changing the beam form of a light beam using a laser beam expander.

The coiled light diffuser of the present invention may be covered with a light-permeable resin. If body fluid such as blood enters the coiled light diffuser or comes into contact with the light diffuser surface, reflectance on the light diffuser surface would vary, which might cause changes in light diffusion efficiency. According to the present invention, a phenomenon in which body fluid enters the light diffuser or comes into contact with the light diffuser surface is explained by noting that infiltration by the body fluid takes place. Infiltration inside the coiled light diffuser by a body fluid such as blood can be prevented by the above film. Examples of a resin used to cover the light diffuser include a transparent silicone resin, a fluorine resin, a polyamide resin, a polyurethane resin, a polyester resin, and a polyolefin resin. The wire rod surface is coated with a resin such that the wire rod becomes covered with the resin film. For example, a wire rod or a coiled light diffuser formed into a coiled shape is immersed in a resin solution and dried such that the coiled light diffuser can be covered with a resin. In addition, a coiled light diffuser covered with a resin can be produced by insert molding. Herein, insert molding refers to a molding method wherein different material parts such as metal parts that are embedded in a plastic compact are first placed in a mold and the mold is filled with a resin via, for example, injection molding. Further, the coiled light diffuser in its entirety is covered with a membranous protection tube made of a resin such that the light diffuser can become covered with a resin. According to the present invention, it can be said that the light diffuser is covered with a light-permeable resin film in any case.

As described above, when the coiled light diffuser of the present invention is used, a light beam transmitted from an optical fiber illuminates the coiled light diffuser of the present invention so as to be diffused. The degree of diffusion of a light beam can he controlled mainly based on the pitch of a portion for diffusing transmitted light, the pitch distribution for the portion for diffusing transmitted light, which is a variable-pitch portion, and the NA of light transmitted from the optical fiber. Specifically, as the pitch of the portion for diffusing transmitted light decreases, the area irradiated with diffused light becomes uniformly wide. In addition, when the portion is a variable-pitch portion, provided that the pitch on the side adjacent to the portion for attachment to an optical fiber is narrow and the pitch of a portion positioned away from the portion for attachment to the optical fiber is wide, the area irradiated with diffused light becomes uniformly wide. Further, when the NA is decreased, the area irradiated with diffused light is extended uniformly. By changing other conditions such as the wire rod diameter, the wire rod section shape, the pitch angle, and the wire rod material, the irradiation area and the uniformity of irradiation can be changed. The coiled light diffuser of the present invention can be designed as described above. Thus, the area irradiated with diffused light can be freely controlled. The present invention also encompasses a method for adjusting the size or the material of the coiled light diffuser so as to control the light irradiation area or the uniformity of light irradiation.

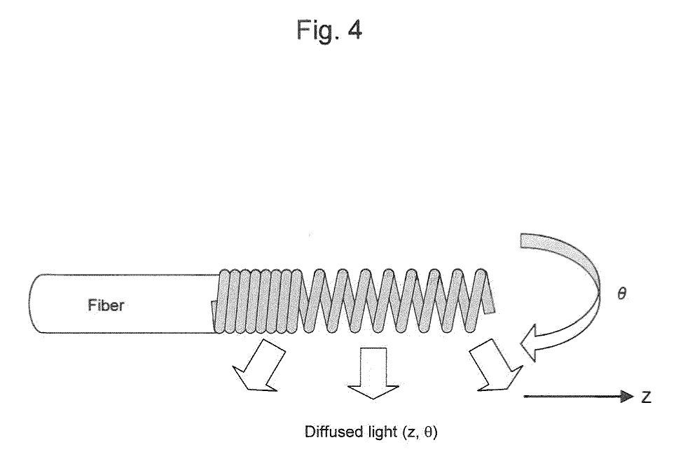

It is possible to examine whether or not a biological tissue can be widely and uniformly irradiated with light transmitted from an optical fiber and diffused from the coiled light diffuser of the present invention in a manner as described below. As shown in FIG. 4, a light beam transmitted from an optical fiber travels in the transmission direction (in the z axis direction) and is circumferentially diffused by a light diffuser, resulting in wide irradiation

The distribution of intensities of light diffused in the z axis direction is examined. Regarding the distribution, the horizontal axis is designated as representing the distance from the fiber end surface (from which a light beam is transmitted) to a given point located along the z axis direction and the vertical axis is designated as representing the diffused light intensity at a given point located a fixed distance away from the fiber centerline. Herein, the distance along the z axis direction is a distance obtained by subtracting the length of a portion overlapping an optical fiber when attached to the optical fiber from the free length of a coiled light diffuser. In a case in which there are few changes in the diffused light intensity at any given point relative to the distance along the z axis direction, a biological tissue can be widely and uniformly irradiated. For instance, in a case in which the distribution of intensities of light diffused in the z axis direction is determined, the minimum diffused light intensity is 10% or more, preferably 25% or more, more preferably 30% or more, and further preferably 50% or more of the maximum diffused light intensity in a given arbitrary-interval corresponding to the area of a biological tissue to be irradiated with a light beam.

The coiled light diffuser of the present invention can diffuse light in all directions when it is used without being deformed. Note that when it is used for a biological tissue such as a lumen tissue of a blood vessel having a complex structure, it is used in a bent state while the tip thereof is in contact with the blood vessel wall in some cases. As described above, it is preferable to use a wire rod comprising an elastic substance for the coiled light diffuser of the present invention, assuming the use of the coiled light diffuser in a bent state. In this case, neighboring coils are narrowly spaced on the bent side of the diffuser while neighboring coils are widely spaced on the side opposite to the bent side. In the bent state, a light beam transmitted from an optical fiber is diffused mainly on the side opposite to the bent side of the coiled light diffuser. Thus, a biological tissue existing on such side can mainly be irradiated with light. As described above, when the coiled light diffuser of the present invention is allowed to come into contact with a biological tissue so as to be bent, irradiation with diffused light can take place in an arbitrary direction. For instance, in a case in which there is a portion to be treated via light beam irradiation only at one site of tissue of the inner wall of a lumen, a specific biological tissue site can be irradiated with light by controlling the bent state of the coiled light diffuser.

The present invention further encompasses an optical fiber that can be used for biological tissue treatment that is a medical optical fiber for in vivo treatment having a tip to which the above coiled light diffuser is attached.

As an optical fiber used in the present invention, a quartz fiber can be used. Optical fibers that can be used include a very thin optical fiber with a diameter of approximately 0.05 to 0.3 mm and an optical fiber having a visible thickness. The optical fiber used can be selected depending on the type of biological tissue to be treated. For instance, when an optical fiber is inserted into a blood vessel and used therein, it can be directly inserted into a blood vessel. Alternatively, a catheter accommodating an optical fiber can be inserted into a blood vessel. A variety of optical fibers with different diameters can be widely used as long as they can transmit a light beam.

Further, the present invention encompasses a device to which the coiled light diffuser of the present invention is attached. The device is a light-diffusing device comprising the coiled light diffuser of the present invention. Herein, such device refers to, for example, a device obtained by assembling a plurality of parts such as a catheter, an endoscopic tool, and a phototherapy device. Examples of such device include a (medical) catheter for in vivo treatment, a catheter-type device for in vivo treatment, and an endoscopic device which comprises an optical fiber having a tip to which the coiled light diffuser of the present invention is attached. Such device is a medical device that can be used for in vivo treatment or diagnosis using a light beam such as a laser light beam. Catheters used in general can be used herein. The catheter diameter and other conditions are not limited. A catheter appropriate for a lesion to be treated can be used. For example, a blood vessel catheter can be used for arteriosclerosis treatment and a urethral catheter can be used for prostate cancer or prostate hypertrophy treatment.

In the device of the present invention, the type of light beam irradiated for treatment or diagnosis is not limited. However, a continuous or pulsed laser light beam or a light beam that is generated by a wavelength-variable optical parametric oscillator (OPO) is preferable. In addition, according to the present invention, the above light beams are collectively referred to as a laser light beam. Wavelengths for irradiation can be adequately determined depending on the contents of treatment. For instance, frequency-doubled laser waves are used in an appropriate manner. Examples of lasers include a semiconductor laser, an excimer dye laser, a dye laser, and a wavelength-variable near-infrared laser. The above light beam may be a pulsed light beam of a pulsed laser or the like or a continuous light beam of a continuous laser or the like. The term “pulse light beam” refers to a light beam with a pulse width of 1 ms or less. In addition, irradiation with continuous light may be intermittently performed using a light chopper such that a pulsed light beam is provided. A light beam used by the device of the present invention is preferably a continuous laser which is a semiconductor laser.

For instance, the coiled light diffuser of the present invention, an optical fiber to which the coiled light diffuser is attached, and a device comprising the coiled light diffuser can be used as described below.

An optical fiber to which the coiled light diffuser of the present invention is attached is placed inside a catheter. One end of the catheter is connected to a light beam generating means (light beam generator). The catheter is inserted into a living body and the light beam transmission portion thereof is delivered to the site of a biological tissue lesion to be treated. Treatment is carried out by transmitting a light beam into the coiled light diffuser.

The coiled light diffuser of the present invention can he used for treatment, including photochemical treatment of biological tissue, biological tissue adhesion, prevention of post-percutaneous transluminal coronary angioplasty restenosis in the cardiovascular system, and myocardial tissue ablation for treatment of arrhythmia and other diseases. According to the present invention, a lesion to be treated can be widely and uniformly irradiated with a light beam such as a laser light beam. Examples of biological tissue include, but are not limited to, tissues of digestive organs, blood vessels, the heart, the lungs, the urethra, and the esophagus. The coiled light diffuser of the present invention can be used in an air environment inside a digestive organ or the like and in a liquid environment inside a blood vessel or the like.

### Examples

The present invention is hereafter described in greater detail with reference to the following examples, although the present invention is not limited thereto.

### Example 1

### Diffused Light Measurement for a Constant-Pitch Coiled Light Diffuser

A coiled light diffuser described below was used.

Wire rod material: Stainless steel SUS 304

Wire rod section: Round shape

Size: Inner diameter: 0.75 mm; Free length: 43 mm (fiber fitting portion: 3 mm; light transmission (irradiation) portion: 40 mm); Wire diameter: 0.18 mm

Pitch (irradiation portion): 0.09 mm, 0.18 mm, and 0.27 mm (three different pitches)

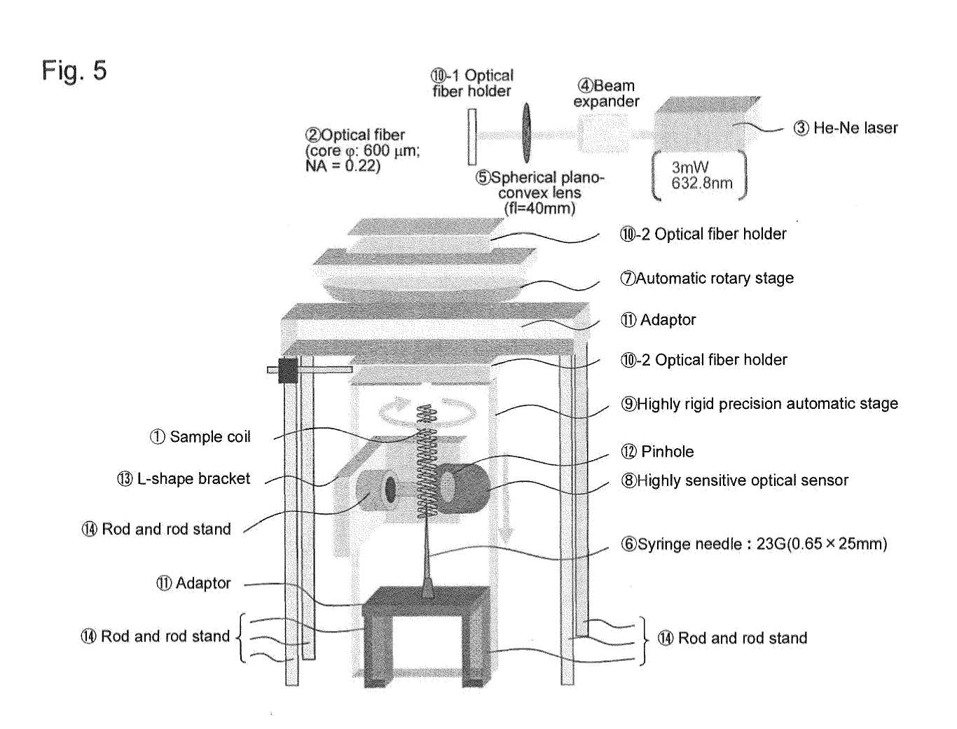

FIG. 5 shows an experimental system. A coiled light diffuser 1 was attached to the tip of an optical fiber 2 (ST600F; NA=0.22; Mitsubishi Cable Industries, Ltd.). An He—Ne laser 3 (632.8 nm. 3 mW; 05LHR111; Melles Griot) was transmitted through the fiber and into the coiled light diffuser. A laser beam expander 4 (LBE-5, SIGMA KOKI Co., Ltd.) and a spherical plano-convex lens 5 (SLB-30-40P, SIGMA KOKI Co., Ltd.) were provided to the system for laser transmission with NA=0.038. A syringe needle 6 (23G; inner diameter: 650 μm; TERUMO Corporation) was placed under the coiled light diffuser for centering. The coiled light diffuser was rotated together with the optical fiber by means of an automatic rotary stage 7 (SGSP-80YAW, SIGMA KOKI Co., Ltd.), followed by measurement of the intensity of light diffused in the direction orthogonal to the laser light irradiation direction with a highly sensitive optical sensor 8 (OP-2VIS; Coherent). Further, the highly-sensitive optical sensor was moved by means of an automatic Z axis stage (highly rigid precision automatic stage) 9 (SGSP26-100(Z); SIGMA KOKI Co., LTD.) for measurement of the intensity of light diffused from the coiled light diffuser in the laser light irradiation direction.

The above experimental equipments (with material model numbers) were used for the purposes described below.

(2) Optical fiber (ST600F; NA=0.22; Mitsubishi Cable Industries, Ltd.) used for irradiation of the inside of a coiled light diffuser with He—Ne laser light.

(3) He—Ne laser (632.8 nm. 3 mW; 05LHR111; Melles Griot) used as a light source for diffused light measurement.

(4) Laser beam expander (LBE-5; SIGMA KOKI Co., Ltd.) used to expand the beam width of He—Ne laser light for light incidence on a fiber for determination of NA.

(5) Spherical plano-convex lens (SLB-30-40P, SIGMA KOKI Co., Ltd.) used to focus light from He—Ne laser light for light incidence on a fiber.

(6) Syringe needle (23G; inner diameter: 650 μm; TERUMO Corporation) used for centering for a rotating coiled,light diffuser.

(7) Automatic rotary stage (SGSP-80YAW, SIGMA KOKI Co., Ltd.) used to rotate a coiled light diffuser upon measurement of light diffused from a sample coil in the direction orthogonal to the laser light emitting direction.

(8) Highly sensitive optical sensor (OP-2VIS; Coherent) used to detect light diffused from a coiled light diffuser.

(9) Highly rigid precision automatic stage (SGSP26-100(Z), SIGMA KOKI Co., Ltd.) used to move a highly-sensitive optical sensor upon measurement of light diffused from a coiled light diffuser in the laser light emitting direction.

(10-1) FC-typc optical fiber holder (FOP-1, SIGMA KOKI Co., Ltd.) used to fix an optical fiber with an FC connector.

(10-2) Optical fiber holder (OFH-1, SIGMA KOKI Co., Ltd.) used to fix an optical fiber.

(11) Acrylic adapter used to support an experimental material.

(12) Pinhole (S71-500; hole diameter σ: 500 μm; Suruga Seiki Co., Ltd.) used to improve spatial resolution upon diffused light measurement.

(13) L-shape bracket (LBR-4053, SIGMA KOKI Co., Ltd.) used to fix a highly sensitive optical sensor on a highly rigid precision automatic stage.

(14) Rod and rod stand (RO and RS series; SIGMA KOKI Co., Ltd.) used to fix an experimental material to a platen.

(15) Zoom-type laser beam expander (LBEZ, SIGMA KOKI Co., Ltd.) used in Example 3.

The NA for light incidence on an optical fiber was adjusted by continuously expanding He—Ne laser light.

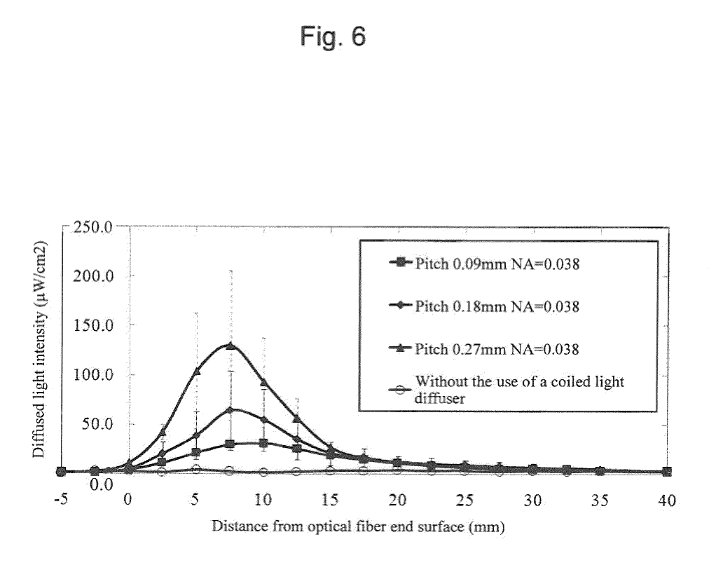

FIG. 6 shows the distribution of intensities of light diffused at different pitches for coiled light diffusers. It was confirmed that the absolute value of diffused light intensity in the case of a constant-pitch coiled light diffuser increased to a greater extent than that obtained without the use of a coiled light diffuser. For each coiled light diffuser, the maximum value of the diffused light intensity was obtained at a distance of approximately 7.5 mm from the fiber tip face. The distance substantially corresponded to the position of light reflection inside the coiled light diffuser, which is calculated based on the angle at which the laser beam spreads. As a result of this experiment, it was confirmed that a decrease in the pitch results in a decrease in the amount of light leaked from a coiled light diffuser, causing laser light to reach the tip of the coiled light diffuser.

### Example 2

### Diffused Light Measurement for a Variable-Pitch Coiled Light Diffuser

Coiled light diffusers described below were used.

Wire rod material: Stainless steel SUS 304

Wire rod section: Round shape

Size: Inner diameter: 0.75 mm; Free length: 43 mm (fiber fitting portion: 3 mm; light transmission (irradiation) portion: 40 mm); Wire diameter: 0.18 mm

Pitch (irradiation portion):
Pitch: 0.09 mm; Length: 10 mm+Pitch: 0.18 mm; Length: 30 mm
Pitch: 0.09 mm; Length: 15 mm+Pitch: 0.18 mm; Length: 25 mm
Pitch: 0.09 mm; Length: 15 mm+Pitch: 0.27 mm; Length 25 mm

An experimental system shown in FIG. 5 was used. A coiled light diffuser 1 was attached to the tip of an optical fiber 2 (ST600F; NA=0.22; Mitsubishi Cable Industries, Ltd.). An He—Ne laser 3 (632.8 nm, 3 mW; 05LHR111; Melles Griot) was transmitted through the fiber and emitted into the coiled light diffuser. A laser beam expander 4 (LBE-5, SIGMA KOKI Co., Ltd.) and a spherical plano-convex lens 5 (SLB-30-40P, SIGMA KOKI Co., Ltd.) were provided to the system for laser transmission with NA=0.038. A syringe needle 6 (23G; inner diameter: 650 μm; TERUMO Corporation) was placed under the coiled light diffuser for centering. The coiled light diffuser was rotated together with the optical fiber by means of an automatic rotary stage 7 (SGSP-80YAW, SIGMA KOKI Co., Ltd.), followed by measurement of the intensity of light diffused in the direction orthogonal to the laser light irradiation direction with a highly sensitive optical sensor 8 (OP-2VIS; Coherent). Further, the highly-sensitive optical sensor was moved by means of an automatic Z axis stage 9 (SGSP26-100(Z); SIGMA KOKI Co., LTD.) for measurement of the intensity of light diffused from the coiled light diffuser in the laser light irradiation direction.

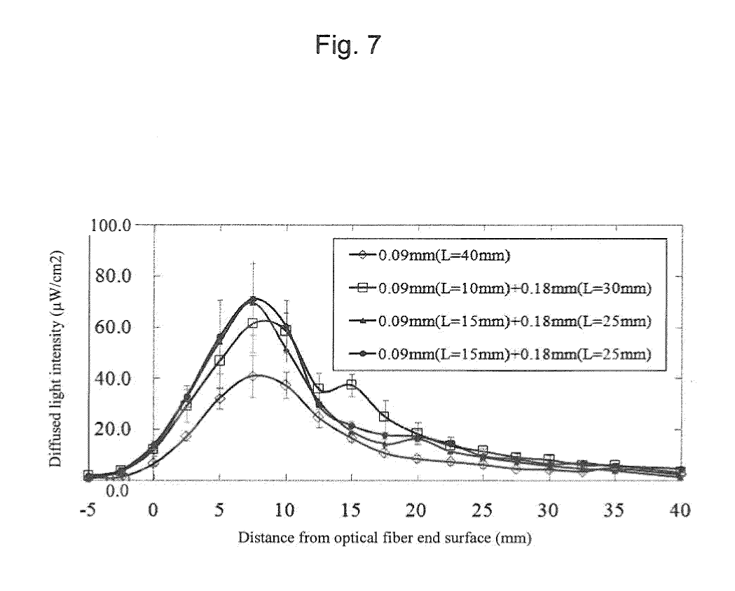

FIG. 7 shows the distribution of diffused light intensities. It was confirmed that, in the case of a variable-pitch coiled light diffuser, laser light illuminated the tip of the coiled light diffuser such that'the absolute value of diffused light intensity increased on the coiled light diffuser tip side to a greater extent than that obtained in the case of a constant-pitch coiled light diffuser. This was probably because coils were loosely spaced on the coiled light diffuser tip side such that light illuminating the tip of a coiled light diffuser was leaked excessively outside the coiled light diffuser. In this Example, it was confirmed that the absolute value of diffused light intensity can be increased even on the coiled light diffuser tip side by designing a coiled light diffuser to have coils spaced at tight to loose pitches from the fiber tip face such that laser light can reach the coiled light diffuser tip.

### Example 3

### Diffused Light Measurement for a Constant-Pitch Coiled Light Diffuser at Different NA Levels of a Transmission Laser Beam

Coiled light diffusers described below were used.

Wire rod material: Stainless steel SUS 304

Wire rod section: Round shape

Size: Inner diameter: 0.75 mm; Free length: 43 mm (fiber fitting portion: 3 mm; irradiation portion for light transmission: 40 mm); Wire diameter: 0.18 mm

Pitch (irradiation portion): 0.09 mm

FIG. 5 shows an experimental system. A coiled light diffuser 1 was attached to the tip of an optical fiber 2 (ST600F; NA=0.22; Mitsubishi Cable Industries, Ltd.). An He—Ne laser 3 (632.8 nm, 3 mW; 05LKHR111; Melles Griot) was transmitted through the fiber and emitted into the coiled light diffuser. A laser beam expander 4 (LBE-5, SIGMA KOKI Co., Ltd.), a spherical plano-convex lens 5 (SLB-30-40P, SIGMA KOKI Co., Ltd.), and a zoom-type laser beam expander 15 (LBEZ, SIGMA KOKI Co., Ltd.) were used for laser transmission with different NA levels (NA=0.038, 0.087, and 0.196). A syringe needle 6 (23G; inner diameter: 650 μm; TERUMO Corporation) was placed under the coiled light diffuser for centering. The coiled light diffuser was rotated together with the optical fiber by means of an automatic rotary stage 7 (SGSP-80YAW, SIGMA KOKI Co., Ltd.), followed by measurement of the intensity of light diffused in the direction orthogonal to the laser light irradiation direction with a highly sensitive optical sensor 8 (OP-2VIS; Coherent). Further, the highly-sensitive optical sensor was moved by means of an automatic Z axis stage 9 (SGSP26-100(Z); SIGMA KOKI Co., LTD.) for measurement of the intensity of light diffused from the coiled light diffuser in the laser light irradiation direction.

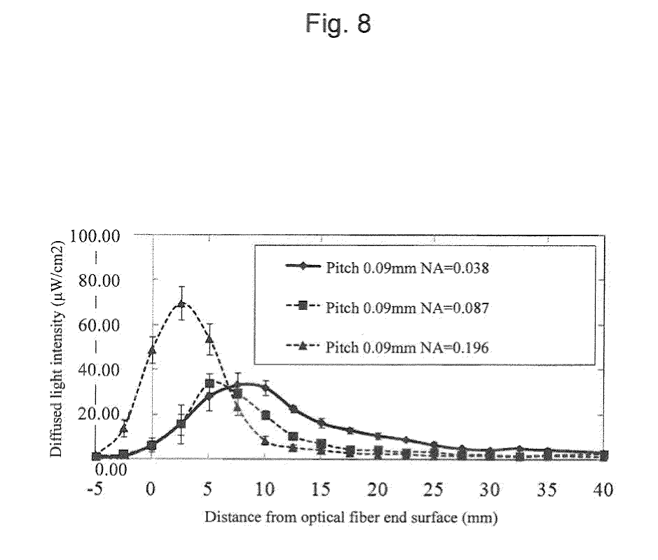

FIG. 8 shows the distribution of diffused light intensities. It was confirmed that when the NA of a transmitted laser beam decreased, the position for detection of the maximum value of diffused light intensity became closer to the coiled light diffuser tip side, resulting in an increase in the absolute value of diffused light intensity even on the coiled light diffuser tip side. This was probably because laser light was incident on a coiled light diffuser in a diagonal direction (tangential direction) such that laser light reach the tip of the coiled light diffuser, resulting in a further decrease in the amount of light leaked outside the coiled light diffuser. In this example, it was confirmed that the absolute value of diffused light intensity can be increased toward the tip of a coiled light diffuser by controlling the NA of a transmitted laser beam

### INDUSTRIAL APPLICABILITY

The coiled light diffuser of the present invention and the light-diffusing device comprising the coiled light diffuser can be used for in vivo treatment using a light beam such as a laser light beam.

All publications, patents, and patent applications cited herein arc incorporated herein by reference in their entirety.

1. A coiled light diffuser consisting of a portion for attachment to an optical fiber and a portion for diffusing light transmitted from an optical fiber, which is attached to a light transmission end of an optical fiber for diffusing light transmitted from the optical fiber in a direction differing from the light transmission direction, and which is formed by coiling a wire rod at least the surface of which comprises a light-reflective metal into a cylindrical shape for the irradiation of a biological tissue with a light beam.

2. The coiled light diffuser according to claim 1, wherein coils of the wire rod that constitute the portion for diffusing light transmitted from an optical fiber are spaced at constant pitches.

3. The coiled light diffuser according to claim 1, which is designed such that coils of the wire rod that constitute the portion for diffusing light transmitted from an optical fiber are spaced at variable pitches, provided that coils are narrowly spaced on the butt side and widely spaced on the tip side.

4. The coiled light diffuser according to claim 1, which comprises a metallic wire rod or a wire rod the surface of which is coated with a light reflective metal.

5. The coiled light diffuser according to claim 1, which is covered with a light-permeable resin film for the prevention of body fluid infiltration.

6. The coiled light diffuser according to claim 1, wherein the tip of the coiled light diffuser accommodates a metal marker used for X-ray radioscopy and/or a biopotential measurement electrode.

7. A light-diffusing device, which comprises the coiled light diffuser according to claim 1.

8. An optical fiber for in vivo treatment, to the tip of which the coiled light diffuser according to claim 1 is attached.
